(19) United States
(12) Patent Application Publication
(10) Pub. No.: US 2024/0141417 A1
(43) Pub. Date: May 2, 2024

(54) MOLECULAR DETECTION SYSTEM AND DETECTION METHOD THEREOF

(71) Applicant: SANSURE BIOTECH INC., Changsha (CN)

(72) Inventors: Lizhong DAI, Changsha (CN); Yaping XIE, Changsha (CN); Tai PANG, Changsha (CN); Jiangang LING, Changsha (CN); Xu TAN, Changsha (CN); Hao YI, Changsha (CN); Zeyu LONG, Changsha (CN)

(21) Appl. No.: 18/404,646

(22) Filed: Jan. 4, 2024

Related U.S. Application Data
(63) Continuation of application No. PCT/CN2022/118224, filed on Sep. 9, 2022.

(30) Foreign Application Priority Data
Feb. 18, 2022 (CN) 202210152697.9

(51) Int. Cl. C12Q 1/6844; B01L 3/00; B01L 7/00; C12Q 1/6806

(52) U.S. Cl. CPC C12Q 1/6844; B01L 3/502753; B01L 7/52; C12Q 1/6806; B01L 2200/04; B01L 2200/10; B01L 2200/16; B01L 2300/027; B01L 2300/0663; B01L 2300/0681; B01L 2300/18

(57) ABSTRACT

Disclosed are a molecular detection system and a detection method thereof. The molecular detection system includes a main control device and a plurality of molecular detection devices. The main control device communicates with each of the plurality of molecular detection devices. The main control device is configured to control each of the plurality of molecular detection devices to perform detection. The main control device includes a display module configured to display detection data of each of the plurality of molecular detection devices. The molecular detection devices may perform different types of detections on different types of samples, which greatly expands the application flexibility and the application scenarios while meeting the detection throughput.

### CROSS-REFERENCE TO RELATED APPLICATIONS

The present application is a continuation application of International Application No. PCT/CN2022/118224 filed on Sep. 9, 2022, which claims priority to Chinese Patent Application No. 202210152697.9 filed on Feb. 18, 2022. Both applications are incorporated herein by reference in their entireties.

### TECHNICAL FIELD

The present disclosure relates to the technical field of biological detection devices, in particular to a molecular detection system and a detection method thereof.

### BACKGROUND

A molecular detection device is used to detect biological samples, mainly including steps of nucleic acid extraction, amplification, detection, etc., in which it is necessary to add samples, and transfer and mix the samples multiple times to complete the above steps of nucleic acid extraction, amplification, detection, etc.

In order to improve the detection capability of the molecular detection device, in the common methods, one method is to increase the detection throughput of a nucleic acid extraction module of the molecular detection device, another method is to increase the number of the nucleic acid extraction modules of the molecular detection device, thereby increasing the detection throughput of the molecular detection device. However, the above methods have the following defects: 1. the single molecular detection device has a complex structure and occupies a large space, which cannot meet the design requirement of miniaturization; 2. after detection parameters of the molecular detection device are set, the same detection can be performed only on the same type of samples, and different types of detections cannot be performed on different types of samples at the same time, which greatly limits the application flexibility and application scenarios of the molecular detection device.

### SUMMARY

Accordingly, it is necessary to provide a molecular detection system and a detection method thereof for overcoming the above-mentioned defects, to address a problem that molecular detection devices in the prior art cannot perform different types of detections on different types of samples at the same time, which greatly limits the application flexibility and application scenarios of the molecular detection device.

A molecular detection system includes a main control device and a plurality of molecular detection devices. The main control device communicates with each of the plurality of molecular detection devices. The main control device is configured to control each of the plurality of molecular detection devices to perform detection. The main control device includes a display module configured to display detection data of each of the plurality of molecular detection devices. Detection performed by the molecular detection devices at least includes nucleic acid extraction, amplification and detection.

As such, the main control device can control the plurality of molecular detection devices to perform detection, and under the control of the main control device, the plurality of molecular detection devices can independently complete the nucleic acid extraction, the amplification and detection, etc., thereby realizing the detection of samples. Since operations of the molecular detection devices are independent of each other, only the main control device performs centralized control of the molecular detection devices, so that the molecular detection devices can perform different types of detections on different types of samples, which greatly expands the application flexibility and the application scenarios while meeting the detection throughput. In addition, the display module of the main control device displays the detection data of each of the molecular detection devices in real time. That is, the detection results of each of the molecular detection devices can be viewed on the display module of the main control device, which is beneficial to comparison, analysis, summarizing, etc., of the detection results, which is convenient and fast.

In one of embodiments, the main control device is further configured to:
in response to receiving a networking command, broadcast a device searching command, receive reply information returned by the molecular detection device according to the device searching command, and add device information of the molecular detection device to a list of devices to be confirmed, where the reply information carries the device information of the molecular detection device; and
broadcast a command for distribution network confirmation, receive distribution network confirmation information returned by the molecular detection device according to the command for distribution network confirmation; and store a device configuration list according to the list of devices to be confirmed in response to receiving the distribution network confirmation information from each of the molecular detection devices in the list of devices to be confirmed.

In one of the embodiments, the main control device is further configured to:
receive a network registration command from the molecular detection device; and
perform network registration processing according to the device configuration list, and return network registration reply information to the molecular detection device,
where in response to the molecular detection device being in the device configuration list, the network registration processing includes allowing the molecular detection device to register network, and setting the molecular detection device to an online state, and the network registration reply information includes successful network registration information. In response to the molecular detection device being not in the device configuration list, the network registration processing includes prohibiting the molecular detection device from registering the network, and the network registration reply information includes information of prohibiting network registration.

In one of the embodiments, the main control device is further configured to:
after setting the molecular detection device to the online state, in response to not receiving a heartbeat packet from the molecular detection device in a heartbeat cycle, disconnect from the molecular detection device, and set the molecular detection device to an offline state.

In one of the embodiments, the main control device is further configured to:
after setting the molecular detection device to the online state, in response to receiving a logout command sent by the molecular detection device, return a logout reply command to the molecular detection device, disconnect from the molecular detection device, and set the molecular detection device to an offline state.

In one of the embodiments, the main control device communicates with each of the plurality of molecular detection devices in a wireless manner.

Or, the main control device communicates with each of the plurality of the molecular detection devices by means of a Registered Jack 45 (RJ45) or a controller area network (CAN) via a wired network.

In one of the embodiments, the main control device regulates detection parameters of each of the plurality of the molecular detection devices to control each of the plurality of the molecular detection devices to perform detection.

Each molecular detection device includes a control module. The control module regulates the detection parameters of the molecular detection device to control the molecular detection device to perform detection.

A priority that the main control device regulates the detection parameters of each molecular detection device is higher than a priority that each molecular detection device's own control module regulates the detection parameters of each molecular detection device.

In one of the embodiments, each molecular detection device includes a loading station and an amplification detection station that are arranged at intervals in a first direction. The molecular detection device includes:
a pushing module controllably movable in the first direction between the loading station and the amplification detection station, the pushing module being configured to receive or unload a reagent device when moving to the loading station;
a pipetting module arranged corresponding to a position between the loading station and the amplification detection station, where the pipetting module is configured to pipet a sample between respective reagent chambers in the reagent device when the pushing module moves between the loading station and the amplification detection station;
an amplification detection module arranged corresponding to the amplification detection station, where the amplification detection module is configured to perform amplification and detection on a reagent containing the sample in the reagent device when the pushing module moves to the amplification detection station; and
a communication module electrically connected to the pushing module, the pipetting module and the amplification detection module, and communicating with the main control device.

In one of the embodiments, the pipetting module includes a connecting part configured to connect with or disconnect from a pipette tip. During a movement of the pushing module between the loading station and the amplification detection station, the connecting part is capable of being alternately aligned with a plurality of reagent chambers of the reagent device in a second direction; the plurality of reagent chambers are configured to preload the sample and/or reagents; and the second direction intersects the first direction.

When the pushing module moves until the connecting part is aligned with any one of the reagent chambers in the second direction, the pipetting module is controllably movable in the second direction to drive the pipette tip on the connecting part to be inserted into or withdraw from a current reagent chamber.

In one of the embodiments, during the movement of the pushing module between the loading station and the amplification detection station, the connecting part is capable of being aligned in the second direction with a tip chamber of the reagent device that is configured to preload the pipette tip.

When the pushing module moves until the connecting part is aligned with the tip chamber in the second direction, the pipetting module is controllably movable in the second direction, so as to drive the connecting part to be inserted into or withdraw from the tip chamber, so that the connecting part picks up the pipette tip in the tip chamber or release the pipette tip on the connecting part into the tip chamber.

In one of the embodiments, during the movement of the pushing module between the loading station and the amplification detection station, the connecting part is capable of being alternately aligned with an injection chamber and a plunger chamber configured to load a plunger of the reagent device in the second direction.

When the pushing module moves until the connecting part is aligned with the plunger chamber in the second direction, the pipetting module is controllably movable in the second direction, thereby driving the connecting part to be inserted into or withdraw from the plunger chamber, to pick up the plunger.

When the pushing module moves until the connecting part is aligned with the injection chamber in the second direction, the pipetting module is controllably movable in the second direction, thereby driving the plunger on the connecting part to be inserted into the injection chamber, to inject the reagent containing the sample in the injection chamber into a reaction tube of the reagent device.

In one of the embodiments, when the pushing module moves to the amplification detection station, the reaction tube of the reagent device is mated with the amplification detection module. The amplification detection module is configured to perform amplification and detection on the reagent containing the sample in the reaction tube of the reagent device.

In one of the embodiments, each molecular detection device further includes a first driving module. The first driving module is drivingly connected to the pipetting module to drive the pipetting module to move in the second direction.

The first driving module is electrically connected to the communication module.

In one of the embodiments, the first driving module includes a first mounting base, a first driving member, a driving wheel, a driven wheel, and a transmission belt.

The pipetting module is movably connected to the first mounting base in the second direction. The first driving member is mounted on the first mounting base, and is electrically connected to the communication module. The driving wheel is drivingly connected to an output shaft of the first driving member. The driven wheel is rotatably connected to the first mounting base and is arranged spaced apart from the driving wheel in the second direction. The transmission belt is sleeved between the driving wheel and the driven wheel, and is fixedly connected to the pipetting module.

In one of the embodiments, the molecular detection device further includes a second driving module. The second driving module is drivingly connected to the pushing module to drive the pushing module to move in the first direction.

The second driving module is electrically connected to the communication module.

In one of the embodiments, the second driving module includes a second mounting base, a second driving member, a screw rod, and a screw rod nut.

The pushing module is movably connected to the second mounting base in the first direction. The screw rod is rotatably connected to the second mounting base around its own axis. An axis of the screw rod is parallel to the first direction. The second driving member is drivingly connected to the screw rod and is electrically connected to the communication module. The screw rod nut is threadedly connected to the screw rod and is fixedly connected to the pushing module.

A molecular detection method applied to the molecular detection system according to any one of the above embodiments, includes steps of:
controlling, by the main control device, each of the plurality of molecular detection devices to perform detection; and
transmitting, by each of the plurality of molecular detection devices, detection data to the main control device, and displaying, by a display module of the main control device, received detection data.

In one of the embodiments, in the step of controlling, by the main control device, each of the plurality of molecular detection devices to perform detection, the detection performed by the molecular detection device includes:
moving a pushing module to a loading station in a first direction, and loading a reagent device on the pushing module, where the reagent device is pre-loaded with a sample, various reagents configured for nucleic acid extraction and pretreatment before detection;
moving the pushing module in the first direction to be between the loading station and an amplification detection station, and pipetting, by a pipetting module, the sample between various reagent chambers in the reagent device, so that the sample is mixed and reacted with the various reagents successively; and
moving the pushing module to the amplification detection station in the first direction, and performing amplification and detection on a reagent containing the sample in the reagent device at the amplification detection station.

### BRIEF DESCRIPTION OF THE DRAWINGS

FIG. 1 is a schematic view of a molecular detection system according to an embodiment of the present disclosure.

FIG. 2 is a schematic view of a main control device of the molecular detection system shown in FIG. 1.

FIG. 3 is a schematic view of a molecular detection device of the molecular detection system shown in FIG. 1.

FIG. 4 is a schematic view of the molecular detection device shown in FIG. 3 (where a casing is omitted).

FIG. 5 is a front view of the molecular detection device shown in FIG. 4.

FIG. 6 is a schematic view of a reagent device according to an embodiment of the present disclosure.

FIG. 7 is a front view of a first driving module of the molecular detection device shown in FIG. 4.

FIG. 8 is a schematic view of a second driving module and a pushing module of the molecular detection device shown in FIG. 4.

FIG. 9 is a flowchart of a molecular detection method of a molecular detection system according to an embodiment of the present disclosure.

FIG. 10 is a detailed flowchart of step S01 of the molecular detection method shown in FIG. 9.

FIG. 11 is a detailed flowchart of step S20 of the molecular detection method shown in FIG. 10.

### DETAILED DESCRIPTIONS OF THE EMBODIMENTS

In order to enable the above objects, features and advantages of the present disclosure more obvious and understandable, the specific embodiments of the present disclosure will be described in detail below with reference to the accompanying drawings. In the following description, many specific details are illustrated in order to aid in understanding of the present disclosure. However, the present disclosure can be implemented in many other ways different from those described herein, and those skilled in the art can make similar improvements without departing from the connotation of the present disclosure. Therefore, the present disclosure is not limited by the specific embodiments disclosed below.

In the description of the present disclosure, it should be understood that orientation or positional conditions indicated by terms “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial”, “circumferential”, etc., are based on orientation or positional relationships shown in the drawings, which are merely to facilitate the description of the present disclosure and simplify the description, not to indicate or imply that the device or elements should have a particular orientation, be constructed and operated in a particular orientation, and therefore cannot be construed as a limitation on the present disclosure.

In addition, the terms “first” and “second” are used for description only, and cannot be understood as indicating or implying relative importance or implicitly indicating the number of technical features indicated. Thus, the features defined with “first” and “second” may include at least one of the features explicitly or implicitly. In the description of the present disclosure, the meaning of “plurality” is at least two, for example, two, three or the like, unless explicitly and specifically defined otherwise.

In the present disclosure, unless explicitly specified and defined otherwise, terms “mounting”, “connecting”, “connected”, and “fixing” should be understood in a broad sense. For example, it may be a fixed connection or a detachable connection, or an integration; may be a mechanical connection or electrical connection; may be a direct connection, or may be a connection through an intermediate medium, may be the communication between two elements or the interaction between two elements, unless explicitly defined otherwise. The specific meanings of the above terms in the present disclosure can be understood by one of those ordinary skills in the art according to specific circumstances.

In the present disclosure, unless expressly specified and defined otherwise, a first feature being “on” or “below” a second feature may mean that the first feature is in direct contact with the second feature, or may mean that the first feature is in indirect contact with the second feature through an intermediate medium. Moreover, the first feature being “above”, “on a top of” and “upside” the second feature may mean that the first feature is directly above or obliquely above the second feature, or simply mean that the level of the first feature is higher than that of the second feature. The first feature being “below”, “under” and “beneath” the second feature may mean that the first feature is directly below or obliquely below the second feature, or simply mean that the level of the first feature is smaller than that of the second feature.

It should be noted that when an element is referred to as being “fixed to” or “provided on” another element, it can be directly on another element or there may be an intermediate element therebetween. When an element is considered to be “connected to” another element, it can be directly connected to another element or there may be an intermediate element therebetween at the same time. The terms “vertical”, “horizontal”, “upper”, “lower”, “left”, “right”, and the like used herein are for illustrative purposes only and are not intended to be the only embodiments.

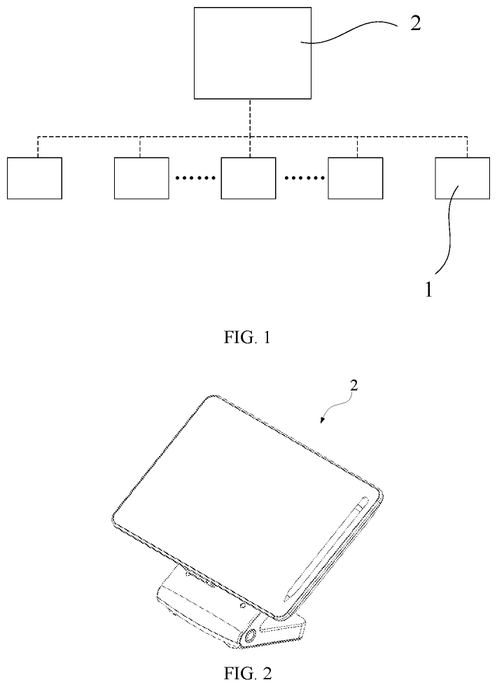

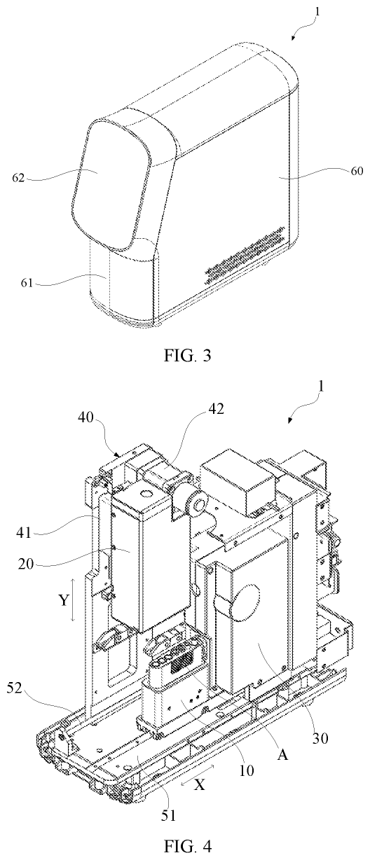

Referring to FIGS. 1, 2, and 3, an embodiment of the present disclosure provides a molecular detection system, including a main control device 2 and a plurality of molecular detection devices 1. The main control device 2 communicates with each of the molecular detection devices 1. The main control device 2 is configured to control each molecular detection device 1 to perform detection. The main control device 2 includes a display module configured to display detection data of each molecular detection device 1. The detection performed by the molecular detection devices 1 at least includes nucleic acid extraction, amplification and detection.

In this way, the main control device 2 can control the plurality of molecular detection devices 1 to perform detection, and under the control of the main control device 2, the plurality of molecular detection devices 1 can independently complete the nucleic acid extraction, the amplification and detection, etc., thereby independently realizing the detection of samples. Since operations of the molecular detection devices are independent of each other, only the main control device performs centralized control of the molecular detection devices, so that the molecular detection devices can perform different types of detections on different types of samples, which greatly expands the application flexibility and the application scenarios while meeting the detection throughput. In addition, the display module of the main control device displays the detection data of each of the molecular detection devices in real time. That is, the detection results of each of the molecular detection devices can be viewed on the display module of the main control device, which is beneficial to comparison, analysis, summarizing, etc., of the detection results, which is convenient and fast.

It should be noted that, under the control of the main control device 2, the plurality of molecular detection devices 1 can independently complete the different molecule detections of the different types of samples, including but not limited to a detection of protein analytes of different samples, a chromosome copy number detection of a gene of interest, a molecular sequencing detection, a nucleic acid extraction, an amplification and detection, etc., which are not limited herein.

Specifically, in an embodiment, the main control device 2 is further configured as follows. In response to receiving, by the main control device 2, a networking command, the main control device 2 is configured to broadcast a device searching command, and receive reply information returned by the molecular detection device according to the device searching command. The reply information carries device information of the molecular detection device. The device information of the molecular detection device is added to a list of devices to be confirmed.

The main control device 2 is configured to broadcast a command for distribution network confirmation, and to receive distribution network confirmation information returned by the molecular detection device according to the command for distribution network confirmation. In response to receiving the distribution network confirmation information from each of the molecular detection devices in the list of devices to be confirmed, a device configuration list is stored according to the list of devices to be confirmed, so that networking between the main control device 2 and each of the molecular detection devices is realized. It should be noted that the networking command can be triggered by an operator touching the display module of the main control device 2. For example, the operator clicks a “Networking” button on the display module, so that the main control device 2 receives the networking command and performs networking.

Further, the main control device 2 is further configured to receive a network registration command from the molecular detection device; perform network registration processing according to the device configuration list, and return network registration reply information to the molecular detection device. In response to the molecular detection device being in the device configuration list, the network registration processing includes allowing the molecular detection device to register network, and setting the molecular detection device to an online state; and the network registration reply information includes successful network registration information. In response to the molecular detection device being not in the device configuration list, the network registration processing includes prohibiting the molecular detection device from registering the network; and the network registration reply information includes information of prohibiting network registration. In this way, each molecular detection device in the device configuration list of the main control device 2 can send the network registration command to the main control device 2. After receiving the network registration command, the main control device 2 allows the molecular detection device to register the network, so that the molecular detection device of which the network registration is successful can transmit data and signals with the main control device 2. For the network registration command sent by the molecular detection device that is not in the device configuration list of the main control device 2, the main control device prohibits such molecular detection device from registering the network.

Further, the main control device 2 is further configured to: after the molecular detection device is set to the online state, in response to not receiving a heartbeat packet from the molecular detection device in a heartbeat cycle, disconnect from the molecular detection device, and set the molecular detection device to an offline state. In this way, it is ensured that the disconnection from the molecular detection device with no data transmission (not performing detection) can be performed in time, so as to avoid occupying the resources of the main control device 2, and enable the molecular detection system to run more smoothly.

Further, the main control device 2 is further configured to: after the molecular detection device is set to the online state, in response to receiving a logout command sent by the molecular detection device, return a logout reply command to the molecular detection device, disconnect from the molecular detection device, and set the molecular detection device to an offline state. In this way, the molecular detection device that does not need to perform detection can actively send the logout command to the main control device 2, so that the main control device 2 can disconnect from the molecular detection device in time, avoiding occupying the resources of the main control device 2, and enabling the molecular detection system to run more smoothly.

Specifically, in an embodiment, the main control device 2 can communicate with each of the molecular detection devices 1 in a wireless manner. Certainly, in another embodiment, the main control device 2 can also communicate with each of the molecular detection devices 1 by means of a Registered Jack 45 (RJ45, a standard 8-bit modular interface) via a wired network. In yet another embodiment, the main control device 2 can also communicate with each of the molecular detection devices 1 via a wired network such as a Controller Area Network (CAN).

Specifically, in an embodiment, the main control device 2 regulates detection parameters of each of the molecular detection devices 1 to control each of the molecular detection devices 1 to perform detection. Each molecular detection device 1 includes a control module. The control module regulates the detection parameters of the molecular detection device 1 to control the molecular detection device 1 to perform detection. A priority that the main control device 2 regulates the detection parameters of each molecular detection device 1 is higher than a priority that each molecular detection device's own control module regulates the detection parameters of each molecular detection device 1. In this way, when the main control device 2 regulates the detection parameters of a certain molecular detection device 1, the control module of the molecular detection device 1 itself is not allowed to regulate the detection parameters (that is, the regulation cannot be performed), preventing the main control device 2 and the control module from regulating the molecular detection device 1 at the same time.

Further, the control module of each molecular detection device 1 includes a display unit. The display unit is configured to display the detection data of the molecular detection device 1 in which the display unit is located. In this way, the detection data of the molecular detection device 1 can be viewed on the display module of the main control device 2, and the detection data can also be viewed on the display unit of the molecular detection device 1.

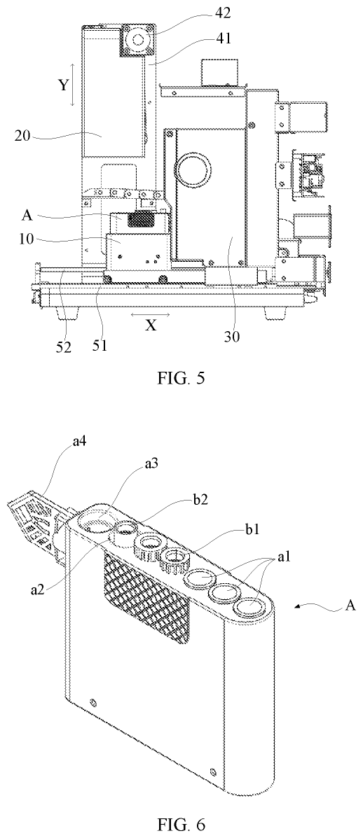

Referring to FIGS. 4 and 5, in an embodiment of the present disclosure, each molecular detection device 1 includes a loading station (i.e., a left end in FIG. 5) and an amplification detection station (i.e., a position of a pushing module 10 shown in FIG. 5) that are arranged at intervals in a first direction X. The molecular detection device 1 includes the pushing module 10, a pipetting module 20, an amplification detection module 30, and a communication module.

The pushing module 10 is controllably movable in the first direction X between the loading station and the amplification detection station. The pushing module 10 is configured to receive or unload a reagent device A when moving to the loading station. The reagent device A is pre-loaded with a sample to be detected and reagents for reacting with the sample. The pipetting module 20 is arranged corresponding to a position between the loading station and the amplification detection station, and is configured to pipet a sample between respective reagent chambers in the reagent device A when the pushing module 10 moves between the loading station and the amplification detection station, so that the sample can be mixed and reacted with the respective reagents to realize the nucleic acid extraction and/or the pretreatment before detection. The amplification detection module 30 is arranged corresponding to the amplification detection station, and is configured to perform amplification and detection on a reagent containing the sample in the reagent device A when the pushing module 10 moves to the amplification detection station. The communication module is electrically connected to the pushing module 10, the pipetting module 20, and the amplification detection module 30, and is communicated with the main control device 2, so that the main control device 2 controls the running of the pushing module 10, the pipetting module 20, and the amplification detection module 30 through the communication module.

The above molecular detection device 1, during the actual detection operation, firstly controls the pushing module 10 to move to the loading station in the first direction X, and loads the reagent device A on the pushing module 10. The reagent device A is pre-loaded with a sample to be detected and various reagents. Then, the pushing module 10 is controlled to move between the loading station and the amplification detection station in the first direction X; and the pipetting module 20 is used to pipet the reagent containing the sample between various reagent chambers in the reagent device A, so that the sample is mixed and reacted with reagents successively, to realize the nucleic acid extraction and/or the pretreatment before detection. Then, the pushing module 10 is controlled to move to the amplification detection station in the first direction X, and the amplification detection module 30 is used to perform amplification and detection on the sample in the reagent device A. After the amplification and detection is completed, the pushing module 10 is controlled to return to the loading station in the first direction X, and the reagent device A that has completed the detection is unloaded, thus completing one detection.

In this way, in the above-mentioned molecular detection device 1, the pushing module 10 drives the reagent device A to move between the loading station and the amplification detection station in the first direction X, so that the nucleic acid extraction, the pretreatment before detection, the amplification and detection can be completed. Comparing to the case that a device positioning module, a Polymerase Chain Reaction (PCR) photoelectric detection and recording module, a magnetic attraction module, a mixing module, a push injection module, etc., are required to be moved or rotated respectively in a related art, the molecular detection device 1 of the present disclosure greatly simplifies the movement process, which is beneficial to simplify the device structure and reduce the required space, and better meets the design requirements of integration and miniaturization.

In an embodiment of the present disclosure, the pipetting module 20 includes a connecting part (not shown) configured to connect with or disconnect from a pipette tip b1 (see FIG. 6). During the movement of the pushing module 10 between the loading station and the amplification detection station, the connecting part of the pipetting module 20 can be alternately aligned with a plurality of reagent chambers a1 of the reagent device A that are configured to preload the samples or the reagents in a second direction Y. The second direction Y intersects the above-mentioned first direction X. Preferably, the second direction Y is perpendicular to the first direction X.

When the pushing module 10 moves until the connecting part is aligned with any one of the reagent chambers a1 in the second direction Y, the pipetting module 20 can be controlled to move in the second direction Y to drive the pipette tip b1 on the connecting part to be inserted into or withdraw from the current reagent chamber a1. In this way, during the actual operation, the pushing module 10 is controlled to move in the first direction X until the connecting part is aligned with the reagent chamber a1 pre-loaded with the sample in the second direction Y, and the pipetting module 20 is controlled to move toward a current reagent chamber a1 in the second direction Y, so that the pipette tip b1 on the connecting part is driven to be inserted into the current reagent chamber a1 and draw the sample. After the drawing is completed, the pipetting module 20 is controlled to move away from the reagent chamber a1 in the second direction Y, thereby driving the pipette tip b1 on the connecting part to withdraw from the current reagent chamber a1. Then, the pushing module 10 is controlled to move in the first direction X until the connecting part is aligned with another reagent chamber a1 in the second direction Y, and the pipetting module 20 is controlled to move toward a current reagent chamber a1 in the second direction Y, thereby driving the pipette tip b1 on the connecting part to be inserted into the current reagent chamber a1 and injecting the drew sample into the current reagent chamber a1, so that the sample is mixed and reacted with the reagent in the current reagent chamber a1. The foregoing processes can be repeated many times in the same manner, so that the sample can be mixed and reacted with each of the reagent solutions successively, so as to complete the nucleic acid extraction and/or the pretreatment before detection.

It should be noted that the current reagent chamber a1 refers to the reagent chamber a1 aligned with the connecting part in the second direction Y. In order to better mix the sample with the reagent in the current reagent chamber a1, the pipetting module 20 can be controlled to repeatedly perform drawing and injecting to achieve the mixing.

Specifically, in an embodiment, during the movement of the pushing module 10 between the loading station and the amplification detection station, the connecting part can be aligned in the second direction Y with a tip chamber of the reagent device A that is configured to preload the pipette tip b1.

When the pushing module 10 moves until the connecting part is aligned with the tip chamber in the second direction Y, the pipetting module 20 can be controlled to move in the second direction Y, so as to drive the connecting part to be inserted into or withdraw from the tip chamber, so that the connecting part can pick up the pipette tip b1 in the tip chamber or release the pipette tip b1 on the connecting part into the tip chamber.

In this way, before pipetting the sample, the connecting part needs to pick up the pipette tip b1. At this time, the pushing module 10 is controlled to move in the first direction X until the connecting part is aligned with the tip chamber in the second direction Y. Then, the pipetting module 20 is controlled to move toward the tip chamber in the second direction Y until the connecting part is inserted into the tip chamber and connected with the pipette tip b1. The pipetting module 20 is controlled to move away from the tip chamber in the second direction Y until the connecting part drives the pipette tip b1 to withdraw from the tip chamber. Then, the pipetting of the reagent containing the sample is carried out in the manner described above.

After the pipetting of the reagent containing the sample is completed, the used pipette tip b1 needs to be released into the tip chamber (to avoid mutual contamination between different reagents). At this time, the pushing module 10 is controlled to move in the first direction X until the connecting part is aligned with the tip chamber in the second direction Y. Then, the pipetting module 20 is controlled to move toward the tip chamber in the second direction Y until the pipette tip b1 is inserted into the tip chamber and separated from the connecting part. Then, the pipetting module 20 is controlled to move away from the tip chamber in the second direction Y until the connecting part withdraws from the tip chamber (while the pipette tip b1 remains in the tip chamber). In this case, if the reagent containing the sample needs to be pipetted again, an unused pipette tip b1 in another tip chamber can be picked up in the same manner, and then the reagent containing the sample can be pipetted.

Specifically, in an embodiment, during the movement of the pushing module 10 between the loading station and the amplification detection station, the connecting part can be alternately aligned with an injection chamber a3 (see FIG. 6) and a plunger chamber a2 (see FIG. 6) for loading a plunger b2 (see FIG. 6) of the reagent device A in the second direction Y.

When the pushing module 10 moves in the first direction X until the connecting part is aligned with the plunger chamber a2 in the second direction Y, the pipetting module 20 can be controlled to move in the second direction Y, thereby driving the connecting part to be inserted into or withdraw from the plunger chamber a2, to pick up the plunger b2 in the plunger chamber a2. When the pushing module 10 moves in the first direction X until the connecting part is aligned with the injection chamber a3 in the second direction Y, the pipetting module 20 can be controlled to move in the second direction Y, thereby driving the plunger b2 on the connecting part to be inserted into the injection chamber a3, and to move along the injection chamber a3 to inject the reagent containing the sample in the injection chamber a3 into a reaction tube a4 (see FIG. 6) of the reagent device A, so as to facilitate subsequent amplification and detection of the reagent containing the sample in the reaction tube a4.

Further, when the pushing module 10 moves to the amplification detection station, the reaction tube a4 of the reagent device A is mated with the amplification detection module 30, and the amplification detection module 30 is configured to perform amplification and detection on the reagent containing the sample in the reaction tube a4.

In this way, after the sample is mixed and reacted with the reagents in the reagent device A, and is pipetted into the injection chamber a3 of the reagent device A, the pushing module 10 moves in the first direction X until the connecting part is aligned with the plunger chamber a2 in the second direction Y. Then, the pipetting module 20 moves toward the plunger chamber a2 in the second direction Y until the connecting part is connected with the plunger b2 in the plunger chamber a2. The pipetting module 20 moves away from the plunger chamber a2 in the second direction Y, thereby driving the plunger b2 to withdraw from the plunger chamber a2 (i.e., the connecting part completes the picking up of the plunger b2). Then, the pushing module 10 moves in the first direction X until the connecting part is aligned with the injection chamber a3 in the second direction Y. The pipetting module 20 moves toward the injection chamber a3 in the second direction Y, and drives the plunger b2 to be inserted into the injection chamber a3 and to move along the injection chamber a3 until the reagent containing the sample in the injection chamber a3 is injected into the reaction tube a4 of the reagent device A. Then, the pipetting module 20 moves away from the injection chamber a3 in the second direction Y, and the connecting part is separated from the plunger b2 and withdraws from the injection chamber a3 (while the plunger b2 remains in the injection chamber a3). The pushing module 10 moves to the amplification detection station in the first direction X, so that the reaction tube a4 of the reagent device A is mated with the amplification detection module 30. At this time, the amplification detection module 30 performs amplification and detection on the reagent containing the sample in the reaction tube a4 of the reagent device A.

It can be understood that the amplification detection module 30 is configured to heat, incubate and cool the reaction tube a4, so that the reagent containing the sample in the reaction tube a4 can be amplified. The amplification detection module 30 is further configured to perform fluorescence detection on the reagent containing the sample in the reaction tube a4.

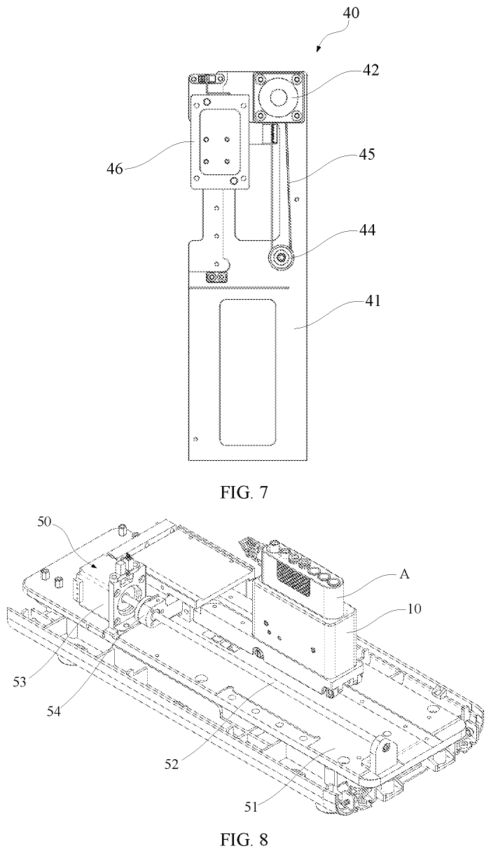

Referring to FIGS. 4, 5, and 7, in an embodiment of the present disclosure, the molecular detection device 1 further includes a first driving module 40. The first driving module 40 is drivingly connected to the pipetting module 20 to drive the pipetting module 20 to move in the second direction Y, so that the pipetting module 20 can complete picking up or releasing the pipette tip b1, drawing or injecting the reagent containing the sample, picking up or releasing the plunger b2, and injecting the reagent containing the sample in the injection chamber a3 into the reaction tube a4 by using the plunger b2, etc. The first driving module 40 is electrically connected to the communication module, so that the main control device 2 controls the first driving module 40 through the communication module.

Specifically, in an embodiment, the first driving module 40 includes a first mounting base 41, a first driving member 42, a driving wheel (not shown), a driven wheel 44, and a transmission belt 45. The pipetting module 20 is movably connected to the first mounting base 41 in the second direction Y. The first driving member 42 is mounted on the first mounting base 41. The driving wheel is drivingly connected to an output shaft of the first driving member 42, so that the driving wheel rotates synchronously with the output shaft of the first driving member 42. The driven wheel 44 is rotatably connected to the first mounting base 41 and is arranged spaced apart from the driving wheel in the second direction Y. The transmission belt 45 is sleeved between the driving wheel and the driven wheel 44, and is fixedly connected to the pipetting module 20, so that the transmission belt 45 can drive the pipetting module 20 to reciprocate in the second direction Y. The first driving member 42 is electrically connected to the communication module, so that the main control device 2 controls the first driving member 42 through the communication module.

In this way, when it is necessary to drive the pipetting module 20 to move in the second direction Y, the first driving member 42 drives the driving wheel to rotate, thereby driving the transmission belt 45 to move forward in sequence between the driving wheel and the driven wheel 44, and then the transmission belt 45 drives the pipetting module 20 to reciprocate in the second direction Y. Optionally, the first driving member 42 may be a motor. It should be noted that, in this embodiment, the movement of the pipetting module 20 in the second direction Y is realized by means of a belt drive. Certainly, in other embodiments, other transmission modes such as air cylinders, screw rods, etc., may also be used, which are not limited herein.

Further, the pipetting module 20 is provided with a first slider 46, and the first mounting base 41 is provided with a first sliding rail (not shown) extending longitudinally in the second direction Y. The first slider 46 is slidably fitted with the first sliding rail. In this way, the movement of the pipetting module 20 relative to the first mounting base 41 in the second direction Y is guided by the movement of the first slider 46 along the first sliding rail.

Referring to FIGS. 4, 5, and 8, in an embodiment of the present disclosure, the molecular detection device 1 further includes a second driving module 50. The second driving module 50 is drivingly connected to the pushing module 10 to drive the pushing module 10 to move in the first direction X, so that the pushing module 10 can reciprocate between the loading station and the amplification detection station, and then can cooperate with the pipetting module 20 and the amplification detection module 30 to complete the nucleic acid extraction, the pretreatment before detection, and the amplification and detection. The second driving module 50 is electrically connected to the communication module, so that the main control device 2 controls the second driving module 50 through the communication module.

Specifically, in an embodiment, the second driving module 50 includes a second mounting base 51, a second driving member 53, a screw rod 52, and a screw rod nut 54.

The pushing module 10 is movably connected to the second mounting base 51 in the first direction X. The screw rod 52 is rotatably connected to the second mounting base 51 around its own axis, and the axis of the screw rod 52 is parallel to the first direction X. The second driving member 53 is drivingly connected to the screw rod 52 to drive the screw rod 52 to rotate around its own axis. The screw rod nut 54 is threadedly connected to the screw rod 52 and is fixedly connected to the pushing module 10. The second driving member 53 is electrically connected to the communication module, so that the main control device 2 controls the second driving member 53 through the communication module.

In this way, when it is necessary to control the pushing module 10 to move in the first direction X, the second driving member 53 drives the screw rod 52 to rotate around its own axis, thereby driving the screw rod nut 54 to move in an axial direction of the screw rod 52 (i.e., the first direction X), and then the screw rod nut 54 drives the pushing module 10 to move in the first direction X. Optionally, the second driving member 53 may be a motor.

Referring to FIG. 2 together, in an embodiment of the present disclosure, the molecular detection device 1 further includes a casing 60. The pushing module 10, the pipetting module 20, and the amplification detection module 30 are all accommodated in the casing 60. In this way, the pushing module 10, the pipetting module 20, and the amplification detection module 30 are integrated in the casing 60 to realize the function of sample-input and result-output, which is beneficial to meet the integrated design requirements.

Specifically, in an embodiment, the casing 60 has an opening (not shown in the figure). When the pushing module 10 moves to the loading station, a carrying position for carrying the reagent device A extends out of the casing 60 from the opening (i.e., get out of the casing), to facilitate unloading the reagent device A on the carrying position, or loading the reagent device A on the carrying position. Further, a door 61 is mounted at the opening of the casing 60. The door 61 is opened during the pushing module 10 moving to the loading station in the first direction X, so that the carrying position of the pushing module 10 can extend out of the casing 60 through the opening. The door 61 is closed during the pushing module 10 moving to the amplification detection station from the loading station in the first direction X. That is, the opening of the casing 60 is closed. It should be noted that the opening and closing of the door 61 can be controlled by means of independent control. Certainly, the opening and closing of the door 61 can also be controlled by means of linkage with the pushing module 10, which is not limited herein.

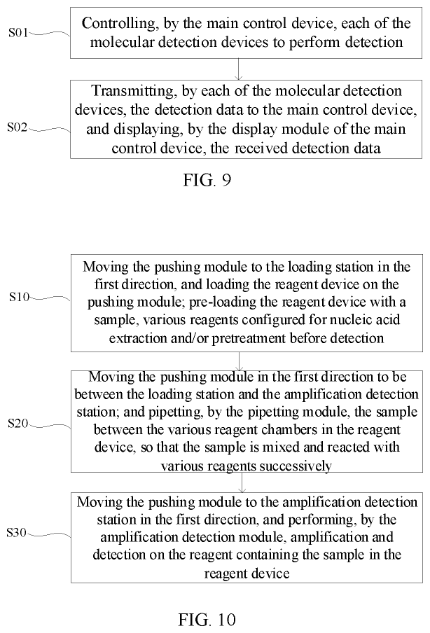

Referring to FIG. 9, based on the above molecular detection system, the present disclosure further provides a molecular detection method, which includes the following steps S01 to S02.

At step S01, the main control device 2 controls each of the molecular detection devices 1 to perform detection.

At step S02, each of the molecular detection devices 1 transmits detection data to the main control device 2, and the display module of the main control device 2 displays the received detection data.

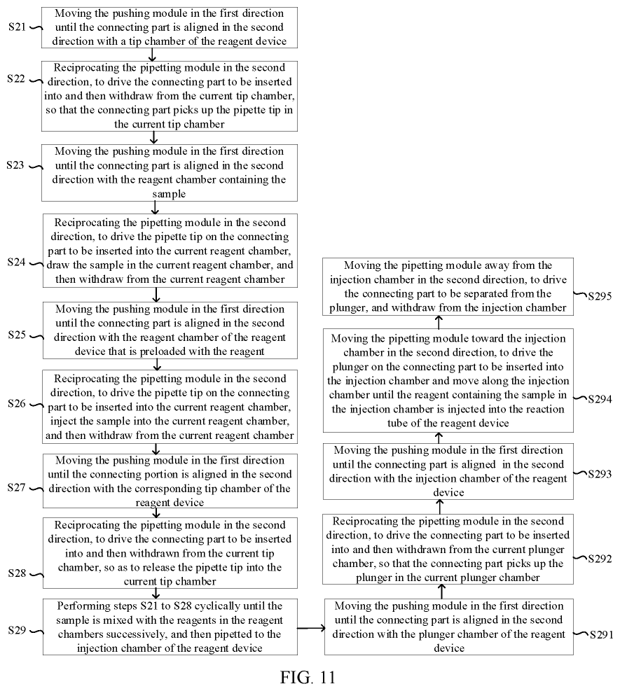

Referring to FIGS. 10 and 11, specifically, in an embodiment, the detection performed by the molecular detection device 1 in step S01 specifically includes the following steps S10 to S30.

At step S10, the pushing module 10 moves to the loading station in the first direction X, and the reagent device A is loaded on the pushing module 10. The reagent device A is pre-loaded with a sample, various reagents configured for nucleic acid extraction and/or pretreatment before detection.

At step S20, the pushing module 10 moves in the first direction X to be between the loading station and the amplification detection station, and the pipetting module 20 pipets the sample between the various reagent chambers in the reagent device A, so that the sample is mixed and reacted with various reagents successively.

At step S30, the pushing module 10 moves to the amplification detection station in the first direction X, and the amplification detection module 30 performs amplification and detection on the reagent containing the sample in the reagent device A.

Specifically, in an embodiment, step S20 specifically includes the following steps S21 to S29.

At step S21, the pushing module 10 moves in the first direction X until the connecting part is aligned with a tip chamber of the reagent device A in the second direction Y.

At step S22, the pipetting module 20 reciprocates in the second direction Y, and drives the connecting part to be inserted into and then withdraw from the current tip chamber, so that the connecting part picks up the pipette tip b1 in the current tip chamber.

At step S23, the pushing module 10 moves in the first direction X until the connecting part is aligned with the reagent chamber a1 containing the sample in the second direction Y.

At step S24, the pipetting module 20 reciprocates in the second direction Y, and drives the pipette tip b1 on the connecting part to be inserted into the current reagent chamber a1, draw the sample in the current reagent chamber a1, and then withdraw from the current reagent chamber a1.

At step S25, the pushing module 10 moves in the first direction X until the connecting part is aligned in the second direction Y with the reagent chamber a1 of the reagent device A that is preloaded with the reagent.

At step S26, the pipetting module 20 reciprocates in the second direction Y, and drives the pipette tip b1 on the connecting part to be inserted into the current reagent chamber a1, inject the sample into the current reagent chamber a1, and then withdraw from the current reagent chamber a1.

At step S27, the pushing module 10 moves in the first direction X until the connecting part is aligned with the corresponding tip chamber of the reagent device A in the second direction Y.

At step S28, the pipetting module 20 reciprocates in the second direction Y, and drives the connecting part to be inserted into and then withdrawn from the current tip chamber, so as to release the pipette tip b1 into the current tip chamber.

At step S29, steps S21 to S28 are performed cyclically until the sample is mixed with the reagents in the reagent chambers a1 successively, and then pipetted to the injection chamber a3 of the reagent device A.

Further, the molecular detection method further includes the following steps S291 to S295 after step S29.

At step S291, the pushing module 10 moves in the first direction X until the connecting part is aligned with the plunger chamber a2 of the reagent device A in the second direction Y.

At step S292, the pipetting module 20 reciprocates in the second direction Y, and drives the connecting part to be inserted into and then withdrawn from the current plunger chamber a2, so that the connecting part picks up the plunger b2 in the current plunger chamber a2;

At step S293, the pushing module 10 moves in the first direction X until the connecting part is aligned with the injection chamber a3 of the reagent device A in the second direction Y.

At step S294, the pipetting module 20 moves toward the injection chamber a3 in the second direction Y, and drives the plunger b2 on the connecting part to be inserted into the injection chamber a3 and moves along the injection chamber a3 until the reagent containing the sample in the injection chamber a3 is injected into the reaction tube a4 of the reagent device A.

At step S295, the pipetting module 20 moves away from the injection chamber a3 in the second direction Y, and drives the connecting part to be separated from the plunger b2 and withdraw from the injection chamber a3.

The technical features of the above-described embodiments can be combined arbitrarily. To simplify the description, not all possible combinations of the technical features in the above embodiments are described. However, all of the combinations of these technical features should be considered as being fallen within the scope of the present disclosure, as long as such combinations do not contradict with each other.

The foregoing embodiments merely illustrate some embodiments of the present disclosure, and descriptions thereof are relatively specific and detailed. However, it should not be understood as a limitation to the patent scope of the present disclosure. It should be noted that, a person of ordinary skill in the art may further make some variations and improvements without departing from the concept of the present disclosure, and the variations and improvements falls in the protection scope of the present disclosure. Therefore, the protection scope of the present disclosure shall be subject to the appended claims.

What is claimed is:

1. A molecular detection system, comprising a main control device and a plurality of molecular detection devices;
wherein the main control device communicates with each of the plurality of molecular detection devices; the main control device is configured to control each of the plurality of molecular detection devices to perform detection; the main control device comprises a display module configured to display detection data of each of the plurality of molecular detection devices; and detection performed by the plurality of molecular detection devices at least comprises nucleic acid extraction, amplification and detection.

2. The molecular detection system according to claim 1, wherein the main control device regulates detection parameters of each of the plurality of the molecular detection devices to control each of the plurality of the molecular detection devices to perform detection;
each molecular detection device comprises a control module; and the control module regulates the detection parameters of the molecular detection device to control the molecular detection device to perform detection,
wherein a priority that the main control device regulates the detection parameters of each molecular detection device is higher than a priority that each molecular detection device's own control module regulates the detection parameters of each molecular detection device.

3. The molecular detection system according to claim 1, wherein the main control device is further configured to:
in response to receiving a networking command, broadcast a device searching command, receive reply information returned by the molecular detection device according to the device searching command, and add device information of the molecular detection device to a list of devices to be confirmed, wherein the reply information carries the device information of the molecular detection device; and
broadcast a command for distribution network confirmation, receive distribution network confirmation information returned by the molecular detection device according to the command for distribution network confirmation; and store a device configuration list according to the list of devices to be confirmed in response to receiving the distribution network confirmation information from each of the plurality of molecular detection devices in the list of devices to be confirmed.

4. The molecular detection system according to claim 3, wherein the main control device is further configured to:
receive a network registration command from the molecular detection device; and
perform network registration processing according to the device configuration list, and return network registration reply information to the molecular detection device,
wherein in response to the molecular detection device being in the device configuration list, the network registration processing comprises allowing the molecular detection device to register network, and setting the molecular detection device to an online state, and the network registration reply information comprises successful network registration information; and
in response to the molecular detection device being not in the device configuration list, the network registration processing comprises prohibiting the molecular detection device from registering the network, and the network registration reply information comprises information of prohibiting network registration.

5. The molecular detection system according to claim 4, wherein the main control device is further configured to:
after setting the molecular detection device to the online state, in response to not receiving a heartbeat packet from the molecular detection device in a heartbeat cycle, disconnect from the molecular detection device, and set the molecular detection device to an offline state.

6. The molecular detection system according to claim 4, wherein the main control device is further configured to:
after setting the molecular detection device to the online state, in response to receiving a logout command sent by the molecular detection device, return a logout reply command to the molecular detection device, disconnect from the molecular detection device, and set the molecular detection device to an offline state.

7. The molecular detection system according to claim 1, wherein the main control device communicates with each of the plurality of molecular detection devices in a wireless manner; or
the main control device communicates with each of the plurality of the molecular detection devices by means of a Registered Jack 45 (RJ45) or a Controller Area Network (CAN) via a wired network.

8. The molecular detection system according to claim 1, wherein each molecular detection device comprises a loading station and an amplification detection station that are arranged at intervals in a first direction; and the molecular detection device comprises:
a pushing module controllably movable in the first direction between the loading station and the amplification detection station, the pushing module being configured to receive or unload a reagent device when moving to the loading station;
a pipetting module arranged corresponding to a position between the loading station and the amplification detection station, wherein the pipetting module is configured to pipet a sample between respective reagent chambers in the reagent device when the pushing module moves between the loading station and the amplification detection station;
an amplification detection module arranged corresponding to the amplification detection station, wherein the amplification detection module is configured to perform amplification and detection on a reagent containing the sample in the reagent device when the pushing module moves to the amplification detection station; and
a communication module electrically connected to the pushing module, the pipetting module, and the amplification detection module, and communicating with the main control device.

9. The molecular detection system according to claim 8, wherein the pipetting module comprises a connecting part configured to connect with or disconnect from a pipette tip;
during a movement of the pushing module between the loading station and the amplification detection station, the connecting part is capable of being alternately aligned with a plurality of reagent chambers of the reagent device in a second direction; the plurality of reagent chambers are configured to preload the sample and/or reagents; the second direction intersects the first direction; and
when the pushing module moves until the connecting part is aligned with any one of the reagent chambers in the second direction, the pipetting module is controllably movable in the second direction to drive the pipette tip on the connecting part to be inserted into or withdraw from a current reagent chamber.

10. The molecular detection system according to claim 9, wherein during the movement of the pushing module between the loading station and the amplification detection station, the connecting part is capable of being aligned in the second direction with a tip chamber of the reagent device that is configured to preload the pipette tip; and
when the pushing module moves until the connecting part is aligned with the tip chamber in the second direction, the pipetting module is controllably movable in the second direction, so as to drive the connecting part to be inserted into or withdraw from the tip chamber, so that the connecting part picks up the pipette tip in the tip chamber or release the pipette tip on the connecting part into the tip chamber.

11. The molecular detection system according to claim 9, wherein during the movement of the pushing module between the loading station and the amplification detection station, the connecting part is capable of being alternately aligned with an injection chamber and a plunger chamber configured to load a plunger of the reagent device in the second direction;
when the pushing module moves until the connecting part is aligned with the plunger chamber in the second direction, the pipetting module is controllably movable in the second direction, thereby driving the connecting part to be inserted into or withdraw from the plunger chamber, to pick up the plunger; and
when the pushing module moves until the connecting part is aligned with the injection chamber in the second direction, the pipetting module is controllably movable in the second direction, thereby driving the plunger on the connecting part to be inserted into the injection chamber, to inject the reagent containing the sample in the injection chamber into a reaction tube of the reagent device.

12. The molecular detection system according to claim 11, wherein when the pushing module moves to the amplification detection station, the reaction tube of the reagent device is mated with the amplification detection module; and the amplification detection module is configured to perform amplification and detection on the reagent containing the sample in the reaction tube of the reagent device.

13. The molecular detection system according to claim 9, wherein each molecular detection device further comprises a first driving module, wherein the first driving module is drivingly connected to the pipetting module to drive the pipetting module to move in the second direction; and
the first driving module is electrically connected to the communication module.

14. The molecular detection system according to claim 13, wherein the first driving module comprises a first mounting base, a first driving member, a driving wheel, a driven wheel, and a transmission belt;
wherein the pipetting module is movably connected to the first mounting base in the second direction;
the first driving member is mounted on the first mounting base, and is electrically connected to the communication module;
the driving wheel is drivingly connected to an output shaft of the first driving member;
the driven wheel is rotatably connected to the first mounting base and is arranged spaced apart from the driving wheel in the second direction; and
the transmission belt is sleeved between the driving wheel and the driven wheel, and is fixedly connected to the pipetting module.

15. The molecular detection system according to claim 8, wherein the molecular detection device further comprises a second driving module;
wherein the second driving module is drivingly connected to the pushing module to drive the pushing module to move in the first direction; and
the second driving module is electrically connected to the communication module.

16. The molecular detection system according to claim 15, wherein the second driving module comprises a second mounting base, a second driving member, a screw rod, and a screw rod nut;
wherein the pushing module is movably connected to the second mounting base in the first direction;
the screw rod is rotatably connected to the second mounting base around its own axis, and an axis of the screw rod is parallel to the first direction;
the second driving member is drivingly connected to the screw rod and is electrically connected to the communication module; and
the screw rod nut is threadedly connected to the screw rod and is fixedly connected to the pushing module.

17. The molecular detection system according to claim 8, wherein each of the plurality of molecular detection devices further includes a casing, the pushing module, the pipetting module, and the amplification detection module are accommodated in the casing.

18. A molecular detection method applied to the molecular detection system according to claim 1, comprising steps of:
controlling, by the main control device, each of the plurality of molecular detection devices to perform detection; and
transmitting, by each of the plurality of molecular detection devices, detection data to the main control device, and displaying, by a display module of the main control device, received detection data.

19. The molecular detection method according to claim 18, wherein in the step of controlling, by the main control device, each of the plurality of molecular detection devices to perform detection, the detection performed by the molecular detection device comprises:
moving a pushing module to a loading station in a first direction, and loading a reagent device on the pushing module, wherein the reagent device is pre-loaded with a sample, various reagents configured for nucleic acid extraction and pretreatment before detection;
moving the pushing module in the first direction to be between the loading station and an amplification detection station, and pipetting, by a pipetting module, the sample between various reagent chambers in the reagent device, so that the sample is mixed and reacted with the various reagents successively; and
moving the pushing module to the amplification detection station in the first direction, and performing amplification and detection on a reagent containing the sample in the reagent device at the amplification detection station.

20. The molecular detection method according to claim 19, wherein the moving the pushing module in the first direction to be between the loading station and an amplification detection station, and pipetting, by a pipetting module, the sample between various reagent chambers in the reagent device, so that the sample is mixed and reacted with the various reagents successively comprises:
moving the pushing module in the first direction until a connecting part is aligned with a tip chamber of the reagent device in a second direction;
reciprocating the pipetting module in the second direction, to drive the connecting part to be inserted into and then withdraw from the current tip chamber, so that the connecting part picks up a pipette tip in the current tip chamber;
moving the pushing module in the first direction until the connecting part is aligned with a first reagent chamber containing the sample in the second direction;
reciprocating the pipetting module in the second direction, to drive the pipette tip on the connecting part to be inserted into the first reagent chamber, draw the sample in the first reagent chamber, and then withdraw from the first reagent chamber;
moving the pushing module in the first direction until the connecting part is aligned in the second direction with a second reagent chamber of the reagent device that is preloaded with a reagent;
reciprocating the pipetting module in the second direction, to drive the pipette tip on the connecting part to be inserted into the second reagent chamber, inject the sample into the second reagent chamber, and then withdraw from the second reagent chamber;
moving the pushing module in the first direction until the connecting part is aligned with the current tip chamber of the reagent device in the second direction;
reciprocating the pipetting module in the second direction, to drive the connecting part to be inserted into and then withdrawn from the current tip chamber, so as to release pipette tip into the current tip chamber; and
performing the above steps cyclically until the sample is mixed with the reagents in reagent chambers successively, and then pipetted to an injection chamber of the reagent device.
